(19) United States
(12) Patent Application Publication
(10) Pub. No.: US 2022/0399090 A1
(43) Pub. Date: Dec. 15, 2022

(54) METHODS AND SYSTEMS FOR DETECTING ADVERSE MEDICAL EVENTS USING ARTIFICIAL INTELLIGENCE

(71) Applicant: DOVEL TECHNOLOGIES, LLC, McLean, VA (US)

(72) Inventor: Rod Fontecilla, McLean, VA (US)

(73) Assignee: DOVEL TECHNOLOGIES, LLC, McLean, VA (US)

(21) Appl. No.: 17/346,414

(22) Filed: Jun. 14, 2021

(51) Int. Cl. G16H 20/10; G06F 40/20; G06N 3/08; G06F 16/30; G06N 3/04; G16H 70/40; G16H 50/70; G16H 40/20; G16H 50/20; G16H 10/60

(52) U.S. Cl. CPC G16H 20/10; G06F 40/20; G06N 3/08; G06F 16/30; G06N 3/0427; G16H 70/40; G16H 50/70; G16H 40/20; G16H 50/20; G16H 10/60

(57) ABSTRACT

Methods and systems are disclosed herein for using artificial intelligence to determine which standardized text description an adverse event reported by a patient may match with. Artificial intelligence/machine learning may be used to determine matches between standardized text descriptions of adverse events and other text descriptions of adverse events (e.g., text descriptions input by patients that have taken a drug). Techniques described herein may improve the functioning of a computing system by allowing it to perform an action that it otherwise could not perform (e.g., determining a standardized text description for an adverse event experienced by a patient).

### BACKGROUND

Developers of vaccines, pharmaceuticals, medical devices, and/or other regulated products will typically conduct several phases of trials before the regulated product may be determined to be safe and effective. During the trials, patients that receive the regulated product may report adverse events (e.g., symptoms or other health outcomes) that they experienced after receiving the regulated product. Adverse event data that is reported by patients may be stored by computing systems such as those used by the Vaccine Adverse Event Reporting System (VAERS) or other adverse event reporting systems. The adverse event data may be used to create labels that indicate adverse events of regulated products so that future patients or medical professionals may be aware of potential risks of receiving regulated products. However, without proper counting and classifying of adverse events, some adverse events may not be added to a label of a regulated product. Failing to include an adverse event on a label may prevent adequate warning for other patients that may wish to take the regulated product.

### SUMMARY

Accordingly, methods and systems for detecting adverse medical events using artificial intelligence are described. Specifically, the methods and systems are described herein for detecting adverse events based on symptoms, feelings, and/or results described by patients. For example, the system may receive descriptions of the symptoms, feelings, and/or results described by patients and automatically correlate these descriptions to one or more adverse events and/or detect unknown adverse events. More specifically, the methods and systems may use artificial intelligence to improve the collection of adverse events associated with vaccines, medicines, medical devices, biologics (e.g., blood components, blood/plasma derivatives, gene therapies, etc), combination products (e.g., pre-filled drug syringes, metered-dose inhalers, nasal spray, etc.), nutritional products (e.g., dietary supplements, medical foods, infant formulas, etc.), cosmetics (e.g., moisturizers, makeup, shampoos, hair dyes, tattoos, etc.), food (e.g., beverages, ingredients added to foods, etc.), and/or other items.

However correlating these descriptions to one or more adverse events and/or detecting unknown adverse events presents several technical hurdles. For example, conventional artificial intelligence systems (e.g., natural language processing) rely on matching one word to another. However, words used (e.g., by patients) to explain adverse events may not match with standardized descriptions for adverse events. In fact, standardized descriptions may appear wholly unrelated to the laymen terminology or the terminology used by a patient. Moreover, the patient may incorrectly describe a symptom or use incorrect terminology. For example, it may be difficult for a patient to explain a symptom or a feeling (e.g., describe what type of “headache” he/she is having), and different patients may use different words for the same adverse event (e.g., one patient's “scratchy throat” may or may not correspond to another patient's “itchy throat”) or assess the same symptom differently (e.g., two patients may have different standards for what constitutes “a medium amount of pain”).

To overcome this technical hurdle, artificial intelligence may be used to improve the collection of adverse events associated with drugs and labels that indicate adverse events. For example, the system may determine matches between standardized text descriptions of adverse events and other text descriptions of adverse events (e.g., text descriptions reported by patients that have taken a drug) that is not hindered by the inconsistencies of patient descriptions. For example, a computing system may use machine learning to generate first vectors that are representative of textual descriptions of adverse events reported by patients. The computing system may also use machine learning to generate second vectors that are representative of medical terminology from a medical dictionary (e.g., the Medical Dictionary for Regulatory Activities). One or more vectors may also be generated based on contextual information associated with a patient (e.g., biographical information of the patient such as height, weight, age, gender, medical history, etc.). The computing system may use the vectors to categorize or correlate patient's descriptions with standardized medical terminology. In addition, using machine learning to generate word vectors and correlate them to other word vectors generated from a medical dictionary may improve the efficiency of the computing system. The use of word vectors generated from a medical dictionary may improve efficiency because it limits the number of comparisons (e.g., between text descriptions reported by patients and standardized medical terminology) the computing system may need to make by the size of the medical dictionary.

A computing system may receive adverse event data corresponding to a drug (e.g., a vaccine, medicine, medical device, biologic, combination product, nutritional product, cosmetic, food, and/or other item). The adverse event data may include text descriptions of adverse events reported by patients that have taken the drug. For example, the computing system may receive adverse event data from a database associated with the Vaccine Adverse Event Reporting System (VAERS), the U.S. Food & Drug Administration Adverse Event Reporting System (FAERS), the Manufacturer and User Facility Device Experience (MAUDE) system, and/or a variety of other adverse event systems. The adverse event data may include a text description from a patient that received a vaccine for the Coronavirus Disease 2019 (COVID-19) and may indicate that the patient experienced a scratchy throat after receiving the vaccine.

The computing system may input the text descriptions into a machine learning model to generate one or more word vectors for each text description. For example, the machine learning model may use the text description indicating that the patient experienced a scratchy throat (after receiving the vaccine) as input to a machine learning model to generate a first word vector. Using word vectors may enable the computing system to more easily compare text descriptions received from a first database (e.g., associated with VAERS) with text descriptions received from a second database. The word vectors may be generated based on contextual information (e.g., medical history of a patient), which may improve the word vectors and the computing system's ability to correlate the text descriptions reported by patients with standardized medical terminology.

The computing system may receive a set of text descriptions, for example, from a second database. The second set of text descriptions may correspond to standardized text descriptions (e.g., used by one or more organizations) for adverse events (e.g., side effects). For example, the computing system may receive text descriptions from the Medical Dictionary for Regulatory Activities (MedDRA). The set of text descriptions may include standardized text descriptions such as “headache,” “throat irritation,” “injection site pain,” etc. The computing system may generate additional word vectors by inputting the second set of text descriptions into the machine learning model. For example, each of the “headache,” “throat irritation,” and “injection site pain” text descriptions may be input into the machine learning model to generate one or more word vectors for each text description. Using standardized text descriptions may improve the efficiency of the computing system (e.g., less processing power may be used) because it may limit the number of items that the computing system has to compare word vectors with (e.g., the word vectors generated from text descriptions associated with VAERS).

The computing system may compare word vectors generated using text descriptions from the first database (e.g., VAERS) with word vectors generated using text descriptions from the second database (e.g., MedDRA) to determine whether there is a match between text descriptions. The computing system may determine a first similarity score indicating a similarity between a first text description corresponding to a first word vector and a second text description corresponding to a second word vector. For example, a word vector generated using the text description “scratchy throat” (e.g., from VAERS) may be compared with a word vector generated using the text description “throat irritation” (e.g., from MedDRA) to generate a similarity score (e.g., using a distance metric such as cosine similarity). For example, the similarity score generated by comparing the word vector for “scratchy throat” and “throat irritation” using a cosine similarity distance metric may be 0.8.

The computing system may compare the similarity score to a threshold similarity score to determine whether the first text description matches the second text description. For example, the threshold similarity score may be 0.75. A similarity score that exceeds the threshold similarity score may be determined to be a match. For example, the similarity score of 0.8 may indicate that “scratchy throat” and “throat irritation” are a match because the similarity score of 0.8 exceeds the threshold similarity score of 0.75. The computing system may generate for display, on a user interface, a recommendation based on comparing the first similarity score to the threshold similarity score. The computing system may generate a recommendation that the term “throat irritation” should be added to a label for the COVID-19 vaccine based on determining that “scratchy throat” and “throat irritation” are a match. Additionally or alternatively, the computing system may correlate text descriptions that match a particular standardized text description so that a frequency of the standardized text description may be properly counted. For example, the computing system may properly aggregate all occurrences of “scratchy throat” with the standardized term “throat irritation” so that counts of the standardized term may be more accurate. This may improve the computing system by enabling more accurate data (e.g., a more accurate number indicating the frequency that patients experienced “throat irritation” after taking a drug) to be stored.

Various other aspects, features, and advantages of the disclosure will be apparent through the detailed description of the disclosure and the drawings attached hereto. It is also to be understood that both the foregoing general description and the following detailed description are examples and not restrictive of the scope of the disclosure. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification “a portion,” refers to a part of, or the entirety of (i.e., the entire portion), a given item (e.g., data) unless the context clearly dictates otherwise.

### BRIEF DESCRIPTION OF THE DRAWINGS

FIG. 1 shows an example natural language processing system for using machine learning to detect adverse medical events, in accordance with some embodiments.

FIG. 2 shows an example flow diagram with actions involved in detecting adverse medical events, in accordance with some embodiments.

FIG. 3A shows an example user interface that may be used to display adverse medical events to a user, in accordance with some embodiments.

FIG. 3B shows an additional example user interface that may be used to display demographics on adverse events for drugs, in accordance with some embodiments.

FIG. 4 shows an example machine learning model, in accordance with some embodiments.

FIG. 5 shows an example computing system that may be used in accordance with some embodiments.

FIG. 6 shows an example flowchart of the actions involved in using machine learning to detect adverse medical events, in accordance with some embodiments.

### DETAILED DESCRIPTION OF THE DRAWINGS

In the following description, for the purposes of explanation, numerous specific details are set forth in order to provide a thorough understanding of the disclosure. It will be appreciated, however, by those having skill in the art, that the disclosure may be practiced without these specific details or with an equivalent arrangement. In other cases, well-known structures and devices are shown in block diagram form to avoid unnecessarily obscuring the disclosure.

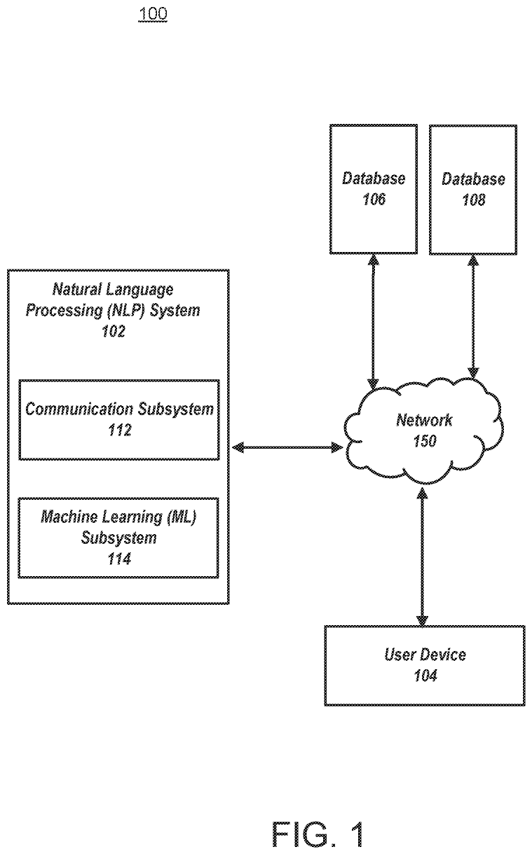

FIG. 1 shows an example computing system 100 for using machine learning or artificial intelligence to improve the collection of adverse events associated with drugs. Throughout this application, the term “drug” may include vaccines, medicines, medical devices, biologics (e.g., blood components, blood/plasma derivatives, gene therapies, etc), combination products (e.g., pre-filled drug syringes, metered-dose inhalers, nasal spray, etc.), nutritional products (e.g., dietary supplements, medical foods, infant formulas, etc.), cosmetics (e.g., moisturizers, makeup, shampoos, hair dyes, tattoos, etc.), food (e.g., beverages, ingredients added to foods, etc.), and/or a variety of other items. A computing system may use machine learning to generate first vectors that are representative of textual descriptions of adverse events reported by patients and generate second vectors that are representative of medical terminology from a medical dictionary (e.g., the Medical Dictionary for Regulatory Activities). One or more vectors may also be generated based on contextual information associated with a patient (e.g., biographical information of the patient such as height, weight, age, gender, medical history, etc.). The computing system may use the vectors to categorize or correlate patient's descriptions with standardized medical terminology. Techniques described herein may improve the functioning of a computing system by allowing the computing system to detect adverse events associated with a drug. The techniques may enable the computing system to correlate multiple different descriptions from patients to corresponding standardized medical terminology. In addition, using machine learning to generate word vectors and correlate them to other word vectors generated from a medical dictionary may improve the efficiency of the computing system. The use of word vectors generated from a medical dictionary may improve efficiency because it limits the number of comparisons (e.g., between text descriptions reported by patients and standardized medical terminology) the computing system may need to make by the size of the medical dictionary. The system 100 may include a natural language processing (NLP) system 102, a user device 104, a database 106, and/or a database 108. The NLP system 102 may include a communication subsystem 112, and/or a machine learning (ML) subsystem 114.

The NLP system 102 may receive adverse event data corresponding to a drug. For example, the communication subsystem 112 may receive the adverse event data from the database 106. The adverse event data may include text descriptions of adverse events reported by patients that have taken the drug. For example, the database 106 may include data associated with the Vaccine Adverse Event Reporting System (VAERS). Adverse event data may include a first plurality of text descriptions. Each text description of the first plurality of text descriptions may indicate an adverse event of the drug. For example, the adverse event data may include a text description from a patient that received a pill to treat COVID-19 and may indicate that the patient experienced a scratchy throat after taking the pill. Text descriptions received by the NLP system 102 may indicate any type of adverse event. An adverse event may be a side effect or other negative effect that a patient experiences after taking a drug. A drug may include any type of medicine in any form (e.g., liquid, tablet, capsule, topical medicine, suppository, drop, inhaler, injection, vaccine, etc.)

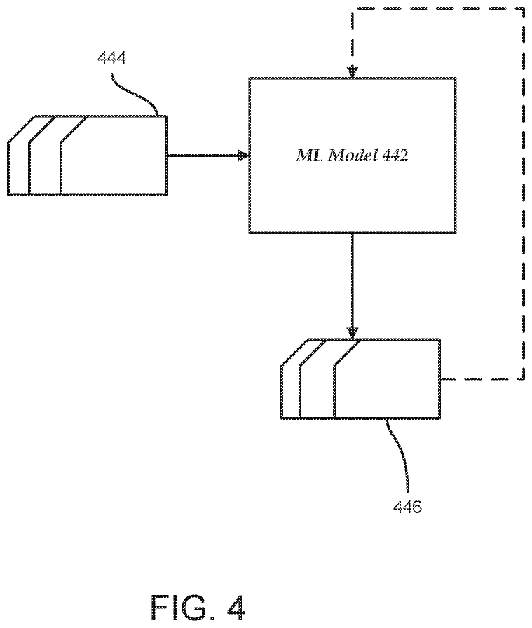

Referring to FIG. 1, the NLP system 102 may input the text descriptions into a machine learning model (e.g., via the ML subsystem 114) to generate a first plurality of word vectors. Each word vector of the first plurality of word vectors may correspond to a text description of the first plurality of text descriptions. For example, the machine learning model may use the text description indicating that the patient experienced a scratchy throat (after receiving the pill) as input to a machine learning model to generate a first word vector. Using word vectors may enable the NLP system 102 to more easily compare text descriptions received from a first database (e.g., associated with VAERS) with text descriptions received from a second database (e.g., associated with MedDRA). The ML subsystem 114 may use any suitable machine learning model (e.g., the machine learning model 442 described below in connection with FIG. 4) to generate one or more word vectors.

The NLP system 102 may receive a second plurality of text descriptions, for example, from the database 108. Each text description of the second plurality of text descriptions may indicate a side effect or other adverse event. Each text description of the second plurality of text descriptions may be associated with a corresponding identification number. The identification number of a text description may be assigned to other text descriptions that are determined to match. For example, the identification number for “throat irritation” may be assigned to the text description “scratchy throat” if the NLP system 102 determines that the two text descriptions match. The second plurality of text descriptions may correspond to standardized text descriptions (e.g., used by one or more organizations) for adverse events (e.g., side effects). For example, the NLP system 102 may receive text descriptions from the Medical Dictionary for Regulatory Activities (MedDRA), a systematically organized computer processable collection of medical terms providing codes, terms, synonyms and/or definitions used in clinical documentation and reporting (e.g., SNOMED clinical terms), the International Statistical Classification of Diseases and Related Health Problems (ICD), and/or the Unified Medical Language System (UMLS) metathesaurus, etc. For example, the second plurality of text descriptions may include standardized text descriptions such as “headache,” “throat irritation,” “injection site pain,” etc.

The NLP system 102 may generate a second plurality of word vectors. Each word vector of the second plurality of word vectors may correspond to a text description of the second plurality of text descriptions. For example, the NLP system 102 may generate the second plurality of word vectors by inputting the second set of text descriptions into the machine learning model. For example, the ML subsystem 114 may input each of the “headache,” “throat irritation,” and “injection site pain” text descriptions into the machine learning model (e.g., the machine learning model 442 described below in connection with FIG. 4) to generate the second plurality of word vectors.

The NLP system 102 may compare word vectors generated using text descriptions from the database 106 (e.g., VAERS) with word vectors generated using text descriptions from the second database (e.g., MedDRA) to determine whether there is a match between text descriptions. The NLP system 102 may determine a first similarity score indicating a similarity between a first text description corresponding to a first word vector and a second text description corresponding to a second word vector. For example, a word vector generated using the text description “scratchy throat” (e.g., from VAERS) may be compared with a word vector generated using the text description “throat irritation” (e.g., from MedDRA) to generate a similarity score (e.g., using a distance metric such as cosine similarity. For example, the similarity score generated by comparing the word vector for “scratchy throat” and “throat irritation” using a cosine similarity distance metric may be 0.8.

The first similarity score may be compared with one or more threshold similarity scores as discussed in more detail below. The NLP system 102 may determine the first similarity score by comparing multiple word vectors with the first word vector and using the word vector (e.g., and its corresponding text description) that is determined to be the closest match for the first word vector (e.g., and the corresponding first text description). This may enable the NLP system 102 to avoid mapping one text description to multiple other text descriptions. For example, if the first text description is “itchy eye,” and there are text descriptions received from the database 108 including “eye irritation,” and “watering eye,” the NLP system 102 may compare a first word vector for “itchy eye” with word vectors for “eye irritation,” and “watering eye” to generate two similarity scores. For example, a first similarity score may indicate a comparison between “itchy eye” and “eye irritation” and a second similarity score may indicate a comparison between “itchy eye” and “watering eye.” The NLP system 102 may determine that “eye irritation” is a closer match to “itchy eye” than “watering eye” (e.g., because the first similarity score is higher) and may determine to use the first similarity score in a comparison with a threshold score. The NLP system 102 may select the highest similarity score to use as the first similarity score (e.g., to use in comparison with the one or more thresholds as discussed in more detail below).

The NLP system 102 may generate, based on a comparison between the first word vector and each word vector of the second plurality of word vectors, a plurality of similarity scores. The NLP system 102 may determine that a first similarity score is higher than any other score of the plurality of similarity scores. In response to determining that the first similarity score is higher than any other score of the plurality of similarity scores, the NLP system 102 may determine that the first similarity score should be used (e.g., as opposed to any other similarity score corresponding to other word vectors) in a comparison with the threshold similarity score.

In some embodiments, the NLP system 102 may determine whether there is an exact match between text descriptions, for example, before generating word vectors and/or similarity scores (e.g., the first similarity score). The NLP system 102 may determine that there is no need to compare word vectors, for example, if there is an exact match between a first text description from the database 106 and a second text description from the database 108. The NLP system 102 may compare the first text description with each text description of the second plurality of text descriptions. Based on comparing the first text description with each text description of the second plurality of text descriptions, the NLP system 102 may determine that the first text description does not match any of the text descriptions of the second plurality of text descriptions. In response to determining that the first text description does not match any of the text descriptions of the second plurality of text descriptions, the NLP system 102 may generate the one or more similarity scores. For example, the NLP system 102 may compare the text description “scratchy throat” with each text description received from the database 108 (e.g., MedDRA text descriptions) and may determine that word vectors should be compared because “scratchy throat” does not match any of the text descriptions received from the database 108.

The NLP system 102 may compare the similarity score to one or more threshold similarity scores to determine whether the first text description matches the second text description. For example, the threshold similarity score may be 0.75. A similarity score that exceeds the threshold similarity score may be determined to be a match. For example, the similarity score of 0.8 may indicate that “scratchy throat” and “throat irritation” are a match because the similarity score of 0.8 exceeds the threshold similarity score of 0.75.

The NLP system 102 may use multiple thresholds to determine whether text descriptions match. For example, if a similarity score is above a high threshold similarity score (e.g., 0.75) the NLP system 102 may determine that the corresponding text descriptions match. If the similarity score is below the high threshold similarity score (e.g., 0.75) and above a medium threshold similarity score (e.g., 0.5), the NLP system 102 may determine that the corresponding text descriptions should be stored or sent for manual review (e.g., by a medical professional). If the similarity score is below the medium threshold similarity score, the NLP system 102 may determine that the corresponding text descriptions (e.g., the first and second text descriptions) do not match. The NLP system 102 may generate, based on an additional word vector of the first plurality of word vectors and the second word vector of the second plurality of word vectors, a second similarity score indicating a similarity level between an additional text description corresponding to the additional word vector and the second text description. The NLP system 102 may determine that the second similarity score fails to exceed the threshold similarity score. Based on determining that the second similarity score fails to exceed the threshold similarity score, the NLP system 102 may generate a data structure comprising the additional text description and the second text description. The NLP system 102 may store the data structure in a queue for review by a medical professional. The NLP system 102 may output (e.g., display) the data structure to a medical professional to enable the medical professional to determine whether the additional text description and the second text description are a match.

The NLP system 102 may determine additional contextual information to include in the data structure, for example, to assist the medical professional in determining whether the additional text description and the second text description are a match. The additional contextual information may include symptoms experienced by a patient associated with the additional text description (e.g., other adverse events reported by the patient that reported the adverse event associated with the additional text description). Based on determining that the second similarity score fails to exceed the threshold similarity score, the NLP system 102 may retrieve contextual information comprising an indication of symptoms experienced by a patient associated with the additional text description. The contextual information may further include biographical information of the patient (e.g., height, weight, age, gender, medical history, etc.). The NLP system may store the contextual information in the data structure.

The NLP system 102 may generate for display, on a user interface, a recommendation. For example, the recommendation may be based on comparing the first similarity score to the threshold similarity score and determining that the first similarity score exceeds the threshold similarity score. A portion of the user interface generated by the NLP system 102 may indicate that the second text description is an adverse event associated with the vaccine and that the second text description does not appear on a label of the vaccine. For example, the NLP system 102 may generate a recommendation that the term “throat irritation” should be added to a label for the COVID-19 vaccine based on determining that “scratchy throat” and “throat irritation” are a match.

Additionally or alternatively, the NLP system 102 may assign an identification number to text descriptions received from the first database (e.g., data stored in VAERS). The identification number that is assigned may be the same identification number of a text description from the second database (e.g., an identification number of a text description in MedDRA) that matches a text description received from the first database. The NLP system 102 may update the data stored in VAERS with the identification number.

The NLP system 102 may recommend adding a text description (e.g., an adverse event) to a label, for example, if more than a threshold number of adverse events of the same type (e.g., matching text descriptions) are determined to exist in adverse event data (e.g., the adverse event data received form the first database). The NLP system 102 may generate, based on a comparison of a word vector with each vector of the first plurality of word vectors, a plurality of similarity scores. The NLP system 102 may determine that more than a threshold number of similarity scores of the plurality of similarity scores exceed the threshold similarity score. In response to determining that more than a threshold number of similarity scores of the plurality of similarity scores exceed the threshold similarity score, the NLP system 102 may generate a recommendation indicating that the second text description should be added to a drug label associated with the drug. For example, if there are more than a threshold number of text descriptions from the first plurality of text descriptions that are determined to match “throat irritation,” the NLP system 102 may generate a recommendation that “throat irritation” be added to a drug label (e.g., if “throat irritation” is not currently on the drug label).

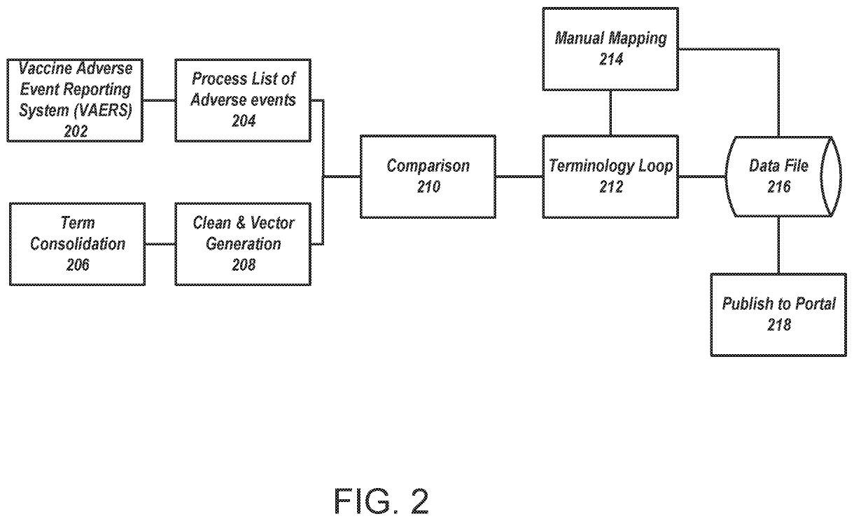

Referring to FIG. 2, an example flow diagram of the steps for using artificial intelligence to detect adverse events is shown. At 202, the NLP system 102 may receive data stored in VAERS (e.g. a list of text descriptions of adverse events). At 204, the NLP system 102 may process the list of adverse events 204. For example, the NLP system 102 may remove special characters, punctuation, split text into individual words, normalize case (e.g., make all letters lower case) or any other suitable processing.

At 206, the NLP system 102 may consolidate terms from one or more databases. For example, the NLP system 102 may consolidate terms from one or more of Medical Dictionary for Regulatory Activities (MedDRA), a systematically organized computer processable collection of medical terms providing codes, terms, synonyms and/or definitions used in clinical documentation and reporting (e.g., SNOMED clinical terms (CL)), the International Statistical Classification of Diseases and Related Health Problems (ICD), and/or the Unified Medical Language System (UMLS) metathesaurus. For example, the NLP system 102 may combine terms from MedDRA and SNOMED CL into one list (e.g., and may remove duplicates). At 208, the NLP system 102 may clean the list (e.g., by removing special characters, stemming, or other suitable edits) and may generate word vectors for each text description in the list.

At 210, the NLP system 102 may compare word vectors generated from the list of adverse events with word vectors generated from the terms consolidated from one or more databases. At 212, the NLP system 102 may loop through each word vector in the list of adverse events to compare with each word vector generated from the terms consolidated from one or more databases.

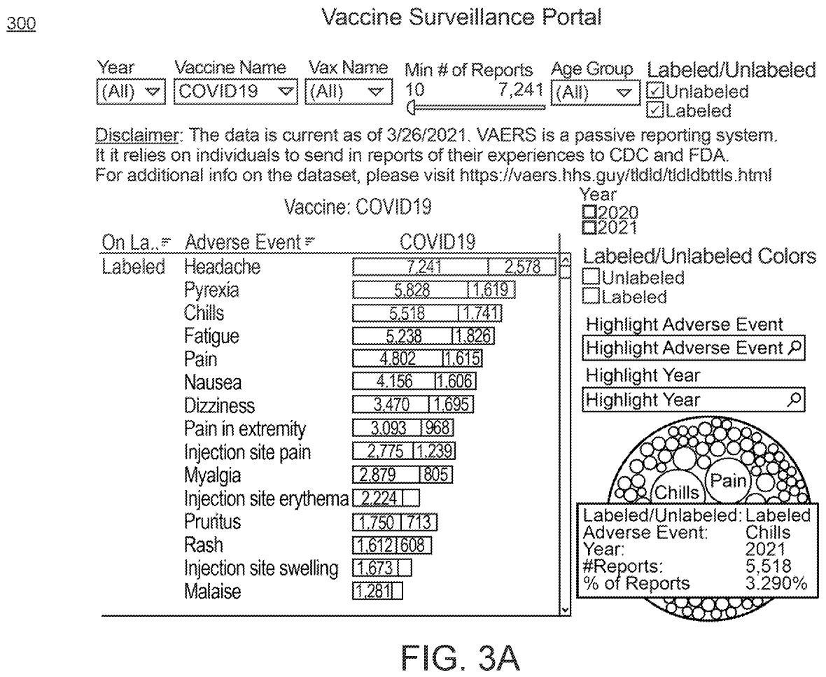

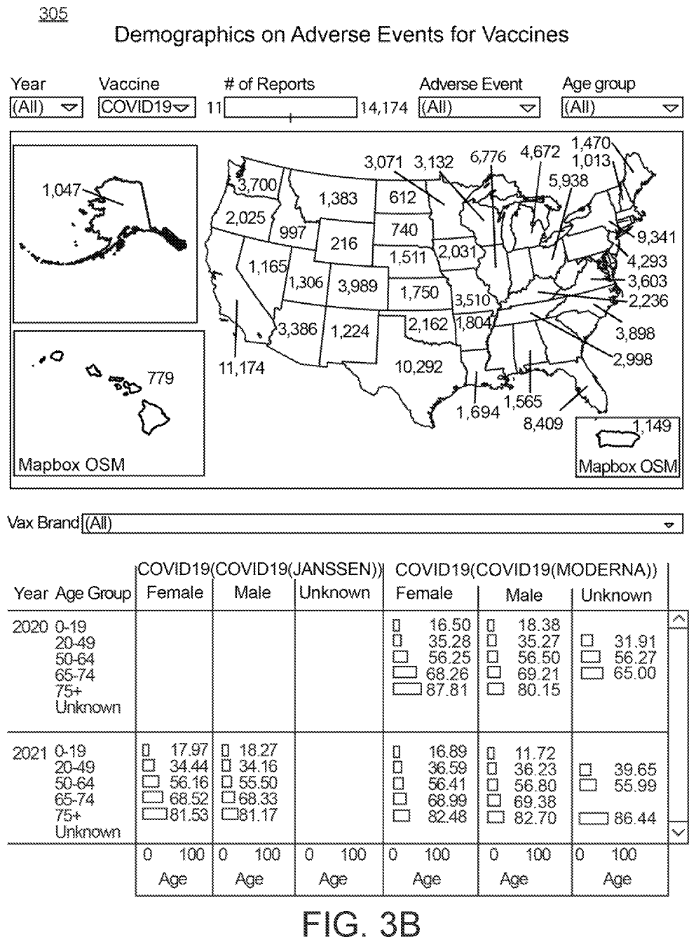

At 214, the NLP system 102 may store text descriptions for manual mapping as discussed above in connection with FIG. 1. At 216, the NLP system 102 may generate a data file. The data file may include information for generating a user interface (e.g., as discussed in more detail below in connection with FIG. 3A). For example, the data file may include a list of adverse events experienced after taking a drug and the number of patients that experienced each adverse event. At 218, the NLP system 102 may publish the contents of the data file generated at 216 to a portal (e.g., a user interface such as the one shown in FIG. 3A).

FIG. 3A shows an example user interface 300 that may be used to display adverse medical events to a user. The user interface may include an element indicating a drug name or disease name. The user interface may include an element indicating a drug that may correspond to the disease. For example, the user interface may include adverse events corresponding to all vaccines for COVID-19. The user interface may indicate an element indicating an age group to which the adverse event data applies (e.g., adults, children, or both). The user interface may include an element which may allow a user to select whether labeled and/or unlabeled data should be displayed in the user interface. The user interface may include an element that includes text descriptions for adverse events (e.g., headache, pyrexia, rash, etc.) experienced by one or more patients that have taken the drug indicated in an element. The user interface may include an element indicating counts corresponding to each adverse event listed in the user interface. For example, there may have been 7,241 patients in 2020 and 2,578 patients in 2021 that reported experiencing a headache after taking a vaccine for COVID-19.

FIG. 3B shows an example user interface 305 for outputting demographics on adverse events for vaccines. The user interface 305 may include one or more geographical regions (e.g., each state in the United States) with a count of one or more adverse events displayed for each region. The user interface 305 may include a table indicating adverse events that are broken down by year, gender, or by other demographic information. A user may be able to adjust an element in the user interface 305 to cause the user interface 305 to update information for different drugs or vaccines.

The user device 104 may be any computing device, including, but not limited to, a laptop computer, a tablet computer, a hand-held computer, smartphone, other computer equipment (e.g., a server or virtual server), including “smart,” wireless, wearable, and/or mobile devices. The user device 104 may be used to report adverse events after taking a drug. The reported adverse events may be store, for example, in the database 106.

The NLP system 102 may include one or more computing devices described above and/or may include any type of mobile terminal, fixed terminal, or other device. For example, the NLP system 102 may be implemented as a cloud computing system and may feature one or more component devices. A person skilled in the art would understand that system 100 is not limited to the devices shown in FIG. 1. Users may, for example, utilize one or more other devices to interact with devices, one or more servers, or other components of system 100. A person skilled in the art would also understand that while one or more operations are described herein as being performed by particular components of the system 100, those operations may, in some embodiments, be performed by other components of the system 100. As an example, while one or more operations are described herein as being performed by components of the NLP system 102, those operations may be performed by components of the user device 104, and/or database 106. In some embodiments, the various computers and systems described herein may include one or more computing devices that are programmed to perform the described functions. Additionally or alternatively, multiple users may interact with system 100 and/or one or more components of system 100. For example, a first user and a second user may interact with the NLP system 102 using two different client devices.

In some embodiments, the NLP system 102 may be part of the user device 104. Providing a message may include outputting a sound, displaying an element in a user interface, vibrating the user device 104, sending information to the user device 104 (e.g., that causes the user device 104 to display a notification), or any other way of providing a notification that may be known to a person of ordinary skill in the art. In some embodiments, the NLP system 102 and the user device 104 may be separate devices and providing a message may include sending, by the NLP system 102, the message to the user device 104.

One or more components of the NLP system 102, user device 104, and/or database 106, may receive content and/or data via input/output (hereinafter “I/O”) paths. The one or more components of the NLP system 102, the user device 104, and/or the database 106 may include processors and/or control circuitry to send and receive commands, requests, and other suitable data using the I/O paths. The control circuitry may include any suitable processing, storage, and/or input/output circuitry. Each of these devices may include a user input interface and/or user output interface (e.g., a display) for use in receiving and displaying data. It should be noted that in some embodiments, the NLP system 102, the user device 104, and/or the database 106-108 may have neither user input interface nor displays and may instead receive and display content using another device (e.g., a dedicated display device such as a computer screen and/or a dedicated input device such as a remote control, mouse, voice input, etc.). Additionally, the devices in system 100 may run an application (or another suitable program). The application may cause the processors and/or control circuitry to perform operations related to using machine learning to determine when notifications should be sent.

One or more components and/or devices in the system 100 may include electronic storages. The electronic storages may include non-transitory storage media that electronically stores information. The electronic storage media of the electronic storages may include one or both of (a) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information obtained from servers, information obtained from client devices, or other information that enables the functionality as described herein.

FIG. 1 also includes a network 150. The network 150 may be the Internet, a mobile phone network, a mobile voice or data network (e.g., a 5G or LTE network), a cable network, a public switched telephone network, a combination of these networks, or other types of communications networks or combinations of communications networks. The devices in FIG. 1 (e.g., NLP system 102, the user device 104, and/or the database 106) may communicate (e.g., with each other or other computing systems not shown in FIG. 1) via the network 150 using one or more communications paths, such as a satellite path, a fiber-optic path, a cable path, a path that supports Internet communications (e.g., IPTV), free-space connections (e.g., for broadcast or other wireless signals), or any other suitable wired or wireless communications path or combination of such paths. The devices in FIG. 1 may include additional communication paths linking hardware, software, and/or firmware components operating together. For example, the NLP system 102, any component of the notification system (e.g., the communication subsystem 112, the ML subsystem 114, and/or the databases 106-108), the user device 104, and/or the database 106 may be implemented by one or more computing platforms.

One or more machine learning models discussed above may be implemented (e.g., in part), for example, as shown in FIG. 4. With respect to FIG. 4, machine learning model 442 may take inputs 444 and provide outputs 446. In one use case, outputs 446 may be fed back to machine learning model 442 as input to train machine learning model 442 (e.g., alone or in conjunction with user indications of the accuracy of outputs 446, labels associated with the inputs, or with other reference feedback information). In another use case, machine learning model 442 may update its configurations (e.g., weights, biases, or other parameters) based on its assessment of its prediction (e.g., outputs 446) and reference feedback information (e.g., user indication of accuracy, reference labels, or other information). In another example use case, where machine learning model 442 is a neural network and connection weights may be adjusted to reconcile differences between the neural network's prediction and the reference feedback. In a further use case, one or more neurons (or nodes) of the neural network may require that their respective errors are sent backward through the neural network to them to facilitate the update process (e.g., backpropagation of error). Updates to the connection weights may, for example, be reflective of the magnitude of error propagated backward after a forward pass has been completed. In this way, for example, the machine learning model 442 may be trained to generate results (e.g., response time predictions, sentiment identifiers, urgency levels, etc.) with better recall and/or precision.

In some embodiments, the machine learning model 442 may include an artificial neural network. In some embodiments, machine learning model 442 may include an input layer and one or more hidden layers. Each neural unit of the machine learning model may be connected with one or more other neural units of the machine learning model 442. Such connections can be enforcing or inhibitory in their effect on the activation state of connected neural units. Each individual neural unit may have a summation function which combines the values of all of its inputs together. Each connection (or the neural unit itself) may have a threshold function that a signal must surpass before it propagates to other neural units. The machine learning model 442 may be self-learning and/or trained, rather than explicitly programmed, and may perform significantly better in certain areas of problem solving, as compared to computer programs that do not use machine learning. During training, an output layer of the machine learning model 442 may correspond to a classification, and an input known to correspond to that classification may be input into an input layer of machine learning model during training. During testing, an input without a known classification may be input into the input layer, and a determined classification may be output. For example, the classification may be an indication of whether an action is predicted to be completed by a corresponding deadline or not. The machine learning model 442 trained by the machine learning subsystem 114 may include one or more embedding layers at which information or data (e.g., any data or information discussed above in connection with FIGS. 1-4A) is converted into one or more vector representations. For example, the embedding layers may be used to generate one or more word vectors based on inputting a text description and/or contextual information into the machine learning model 442.

The machine learning model 442 may be structured as a factorization machine model. The machine learning model 442 may be a non-linear model and/or supervised learning model that can perform classification and/or regression. For example, the machine learning model 442 may be a general-purpose supervised learning algorithm that the system uses for both classification and regression tasks.

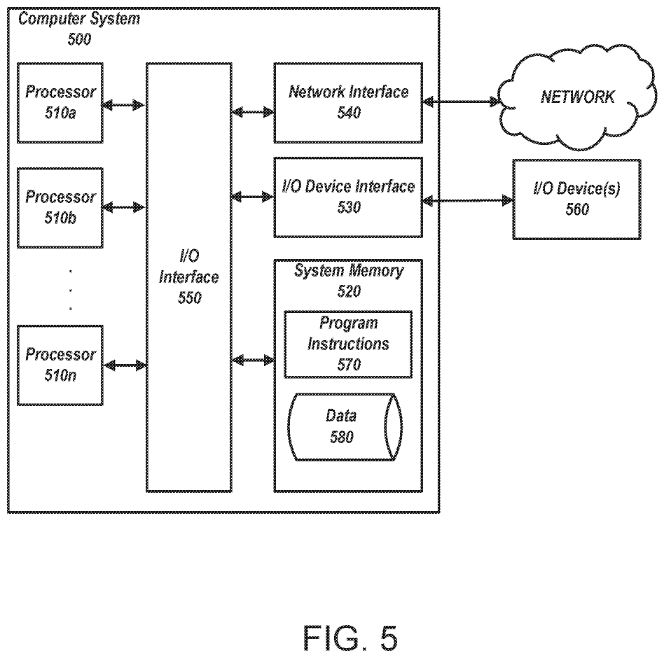

FIG. 5 is a diagram that illustrates an exemplary computing system 500 in accordance with embodiments of the present technique. Various portions of systems and methods described herein, may include or be executed on one or more computer systems similar to computing system 500. Further, processes and modules described herein may be executed by one or more processing systems similar to that of computing system 500.

Computing system 500 may include one or more processors (e.g., processors 510a-510n) coupled to system memory 520, an input/output I/O device interface 530, and a network interface 540 via an input/output (I/O) interface 550. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 500. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 520). Computing system 500 may be a units-processor system including one processor (e.g., processor 510a), or a multi-processor system including any number of suitable processors (e.g., 510a-510n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 500 may include a plurality of computing devices (e.g., distributed computer systems) to implement various processing functions.

I/O device interface 530 may provide an interface for connection of one or more I/O devices 560 to computer system 500. I/O devices may include devices that receive input (e.g., from a user) or output information (e.g., to a user). I/O devices 560 may include, for example, a graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 560 may be connected to computer system 500 through a wired or wireless connection. I/O devices 560 may be connected to computer system 500 from a remote location. I/O devices 560 located on remote computer system, for example, may be connected to computer system 500 via a network and network interface 540.

Network interface 540 may include a network adapter that provides for connection of computer system 500 to a network. Network interface may 540 may facilitate data exchange between computer system 500 and other devices connected to the network. Network interface 540 may support wired or wireless communication. The network may include an electronic communication network, such as the Internet, a local area network (LAN), a wide area network (WAN), a cellular communications network, or the like.

System memory 520 may be configured to store program instructions 570 or data 580. Program instructions 570 may be executable by a processor (e.g., one or more of processors 510a-510n) to implement one or more embodiments of the present techniques. Instructions 570 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 520 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 520 may include a non-transitory computer readable storage medium that may have program instructions stored thereon that are executable by a computer processor (e.g., one or more of processors 510a-510n) to cause the subject matter and the functional operations described herein. A memory (e.g., system memory 520) may include a single memory device and/or a plurality of memory devices (e.g., distributed memory devices).

I/O interface 550 may be configured to coordinate I/O traffic between processors 510a-510n, system memory 520, network interface 540, I/O devices 560, and/or other peripheral devices. I/O interface 550 may perform protocol, timing, or other data transformations to convert data signals from one component (e.g., system memory 520) into a format suitable for use by another component (e.g., processors 510a-510n). I/O interface 550 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 500 or multiple computer systems 500 configured to host different portions or instances of embodiments. Multiple computer systems 500 may provide for parallel or sequential processing/execution of one or more portions of the techniques described herein.

Those skilled in the art will appreciate that computer system 500 is merely illustrative and is not intended to limit the scope of the techniques described herein. Computer system 500 may include any combination of devices or software that may perform or otherwise provide for the performance of the techniques described herein. For example, computer system 500 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 500 may also be connected to other devices that are not illustrated and/or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. In some embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 500 may be transmitted to computer system 500 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present disclosure may be practiced with other computer system configurations.

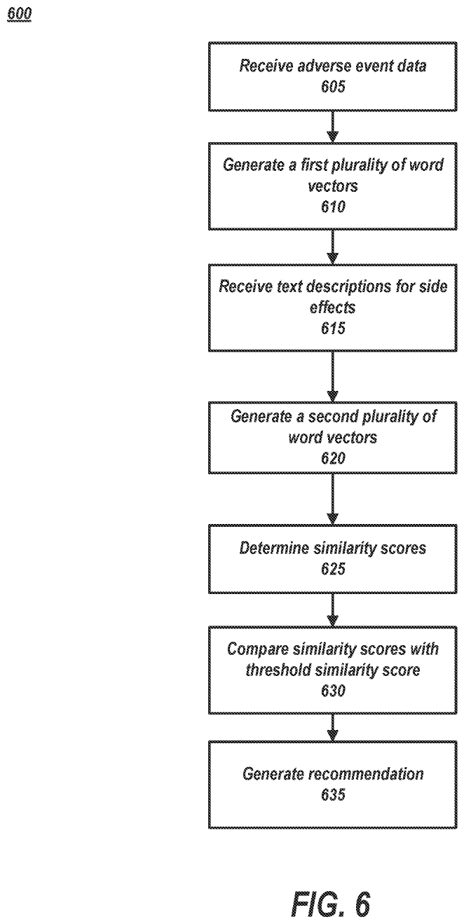

FIG. 6 shows an example flowchart of the actions involved in using machine learning to determine adverse events associated with drugs. For example, process 600 may represent the actions taken by one or more devices shown in FIGS. 1-5 and described above. At 605, NLP system 102 (e.g., using one or more components in system 100 (FIG. 1) and/or computer system 500 via network interface 540 (FIG. 5)) receives adverse event data. The adverse event data may be received from the database 106. The adverse event data may correspond to a drug (e.g., a vaccine, or other medicine). The adverse event data may include a first plurality of text descriptions. Each text description may indicate an adverse event (e.g., as described in more detail above) that has been experienced by a patient that has taken the drug.

At 610, NLP system 102 (e.g., using one or more components in system 100 (FIG. 1) and/or computing system 500 via one or more processors 510a-510n, I/O interface 550, and/or system memory 520 (FIG. 5)) generates a first plurality of word vectors. The NLP system 102 may input the first plurality of text descriptions into a machine learning model (e.g., the model 442 described in connection with FIG. 4) to generate the word vectors. One or more word vectors may be generated for each text description of the first plurality of text descriptions. Additionally or alternatively, one or more vectors may also be generated based on contextual information associated with a patient (e.g., biographical information of the patient such as height, weight, age, gender, medical history, etc.). For example, NLP system 102 may use the vectors to categorize or correlate patient's descriptions with standardized medical terminology.

At 615, NLP system 102 (e.g., using one or more components in system 100 (FIG. 1) and/or computing system 500 via one or more processors 510a-510n (FIG. 5)) receives text descriptions for side effects. The NLP system 102 may receive a second plurality of text descriptions from the database 108. Each text description of the second plurality of text descriptions may indicate a standardized medical terminology (e.g., each text description of the second plurality of text descriptions may be a term from the Medical Dictionary for Regulatory Activities).

At 620, NLP system 102 (e.g., using one or more components in system 100 (FIG. 1) and/or computing system 500 via one or more processors 510a-510n and system memory 520 (FIG. 5)) generates a second plurality of word vectors. Each word vector of the second plurality of word vectors may correspond to a text description of the second plurality of text descriptions.

At 625, NLP system 102 (e.g., using one or more components in system 100 (FIG. 1) and/or computing system 500 (FIG. 5)) determines similarity scores by comparing word vectors in the first plurality of word vectors with word vectors in the second plurality of word vectors. For example, the NLP system 102 may determine, based on a comparison of a first word vector of the first plurality of word vectors with a second word vector of the second plurality of word vectors, a first similarity score indicating a similarity between a first text description corresponding to the first word vector and a second text description corresponding to the second word vector.

At 630, NLP system 102 (e.g., using one or more components in system 100 (FIG. 1) and/or computing system 500 via one or more processors 510a-510n and system memory 520 (FIG. 5)) compares similarity scores with one or more threshold similarity scores to determine whether the first text description matches the second text description.

At 635, NLP system 102 (e.g., using one or more components in system 100 (FIG. 1) and/or computing system 500 via the network interface 540 (FIG. 5) generates a recommendation. The recommendation may indicate, for example, that the first text description corresponding to the first word vector should be added to a label for the drug.

It is contemplated that the actions or descriptions of FIG. 6 may be used with any other embodiment of this disclosure. In addition, the actions and descriptions described in relation to FIG. 6 may be done in alternative orders or in parallel to further the purposes of this disclosure. For example, each of these actions may be performed in any order, in parallel, or simultaneously to reduce lag or increase the speed of the system or method. Furthermore, it should be noted that any of the devices or equipment discussed in relation to FIGS. 1-5 could be used to perform one or more of the actions in FIG. 6.

In block diagrams, illustrated components are depicted as discrete functional blocks, but embodiments are not limited to systems in which the functionality described herein is organized as illustrated. The functionality provided by each of the components may be provided by software or hardware modules that are differently organized than what is presently depicted, for example such software or hardware may be intermingled, conjoined, replicated, broken up, distributed (e.g., within a data center or geographically), or otherwise differently organized. The functionality described herein may be provided by one or more processors of one or more computers executing code stored on a tangible, non-transitory, machine readable medium. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several disclosures. Rather than separating those disclosures into multiple isolated patent applications, applicants have grouped these disclosures into a single document because their related subject matter lends itself to economies in the application process. However, the distinct advantages and aspects of such disclosures should not be conflated. In some cases, embodiments address all of the deficiencies noted herein, but it should be understood that the disclosures are independently useful, and some embodiments address only a subset of such problems or offer other, unmentioned benefits that will be apparent to those of skill in the art reviewing the present disclosure. Due to costs constraints, some features disclosed herein may not be presently claimed and may be claimed in later filings, such as continuation applications or by amending the present claims. Similarly, due to space constraints, neither the Abstract nor the Summary sections of the present document should be taken as containing a comprehensive listing of all such disclosures or all aspects of such disclosures.

It should be understood that the description and the drawings are not intended to limit the disclosure to the particular form disclosed, but to the contrary, the intention is to cover all modifications, equivalents, and alternatives falling within the spirit and scope of the present disclosure as defined by the appended claims. Further modifications and alternative embodiments of various aspects of the disclosure will be apparent to those skilled in the art in view of this description. Accordingly, this description and the drawings are to be construed as illustrative only and are for the purpose of teaching those skilled in the art the general manner of carrying out the disclosure. It is to be understood that the forms of the disclosure shown and described herein are to be taken as examples of embodiments. Elements and materials may be substituted for those illustrated and described herein, parts and processes may be reversed or omitted, and certain features of the disclosure may be utilized independently, all as would be apparent to one skilled in the art after having the benefit of this description of the disclosure. Changes may be made in the elements described herein without departing from the spirit and scope of the disclosure as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing actions A, B, C, and D) encompasses both all such attributes or functions being mapped to all such objects and subsets of the attributes or functions being mapped to subsets of the attributes or functions (e.g., both all processors each performing actions A-D, and a case in which processor 1 performs action A, processor 2 performs action B and part of action C, and processor 3 performs part of action C and action D), unless otherwise indicated. Further, unless otherwise indicated, statements that one value or action is “based on” another condition or value encompass both instances in which the condition or value is the sole factor and instances in which the condition or value is one factor among a plurality of factors. The term “each” is not limited to “each and every” unless indicated otherwise. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

The above-described embodiments of the present disclosure are presented for purposes of illustration and not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

The present techniques will be better understood with reference to the following enumerated embodiments:
1. A method comprising: receiving adverse event data corresponding to a drug, wherein the adverse event data comprises a first plurality of text descriptions; generating a first plurality of word vectors; receiving a second plurality of text descriptions; generating a second plurality of word vectors; determining a first similarity score indicating a similarity between a first text description corresponding to the first word vector and a second text description corresponding to the second word vector; comparing the first similarity score to a threshold similarity score; and based on comparing the first similarity score to the threshold similarity score, generating a recommendation.
2. The method of any of the preceding embodiments, further comprising: generating, based on an additional word vector of the first plurality of word vectors and the second word vector of the second plurality of word vectors, a second similarity score indicating a similarity level between an additional text description corresponding to the additional word vector and the second text description; determining that the second similarity score fails to exceed the threshold similarity score; based on determining that the second similarity score fails to exceed the threshold similarity score, generating a data structure comprising the additional text description and the second text description; and storing the data structure in a queue for review by a medical professional.
3. The method of any of the preceding embodiments, wherein generating a data structure comprising the additional text description and the second text description comprises determining that the second similarity score exceeds a second threshold similarity score.
4. The method of any of the preceding embodiments, further comprising: based on determining that the second similarity score fails to exceed the threshold similarity score, retrieving contextual information comprising an indication of symptoms experienced by a user associated with the additional text description, wherein the contextual information further comprises biographical information of the user; and storing the contextual information in the data structure.
5. The method of any of the preceding embodiments, further comprising: generating, based on a comparison of the second word vector with each vector of the first plurality of word vectors, a plurality of similarity scores; determining that more than a threshold number of similarity scores of the plurality of similarity scores exceed the threshold similarity score; and in response to determining that more than a threshold number of similarity scores of the plurality of similarity scores exceed the threshold similarity score, generating a recommendation indicating that the second text description should be added to a drug label associated with the drug.
6. The method of any of the preceding embodiments, further comprising: comparing the first text description with each text description of the second plurality of text descriptions; based on comparing the first text description with each text description of the second plurality of text descriptions, determining that the first text description does not match any of the text descriptions of the second plurality of text descriptions; and in response to determining that the first text description does not match any of the text descriptions of the second plurality of text descriptions, determining the first similarity score.
7. The method of any of the preceding embodiments, wherein storing an indication that the first text description matches the second text description comprises: generating, based on a comparison between the first word vector and each word vector of the second plurality of word vectors, a plurality of similarity scores; determining that the first similarity score is higher than any other score of the plurality of similarity scores; and in response to determining that the first similarity score is higher than any other score of the plurality of similarity scores, storing an indication that the first text description matches the second text description.
8. The method of any of the preceding embodiments, wherein a portion of the user interface indicates that the second text description is an adverse event a patient experienced after taking the drug and that the second text description does not appear on a label of the drug.
9. A tangible, non-transitory, machine-readable medium storing instructions that, when executed by a data processing apparatus, cause the data processing apparatus to perform operations comprising those of any of embodiments 1-8.
10. A system comprising: one or more processors; and memory storing instructions that, when executed by the processors, cause the processors to effectuate operations comprising those of any of embodiments 1-8.
11. A system comprising means for performing any of embodiments 1-8.

What is claimed is:

1. A system for using machine learning and natural language processing to determine which adverse events indicated in a database should be added to a vaccine label, the system comprising:
a first database comprising a plurality of adverse event data corresponding to a plurality of vaccines;
a second database comprising a second plurality of text descriptions, wherein each text description of the second plurality of text descriptions indicates a side effect, and wherein each text description of the second plurality of text descriptions is associated with a corresponding identification number; and
one or more processors and computer program instructions that, when executed, cause the one or more processors to perform operations comprising:
receiving, from the first database, adverse event data of the plurality of adverse event data corresponding to a vaccine of the plurality of vaccines, wherein the adverse event data comprises a first plurality of text descriptions, wherein each text description of the first plurality of text descriptions indicates a side effect of the vaccine;
generating, based on inputting the first plurality of text descriptions and contextual information into a machine learning model, a first plurality of word vectors, wherein each word vector of the first plurality of word vectors corresponds to a text description of the first plurality of text descriptions;
receiving, from the second database, the second plurality of text descriptions;
generating a second plurality of word vectors, wherein each word vector of the second plurality of word vectors corresponds to a text description of the second plurality of text descriptions;
determining, based on a comparison of a first word vector of the first plurality of word vectors with a second word vector of the second plurality of word vectors, a first similarity score indicating a similarity between a first text description corresponding to the first word vector and a second text description corresponding to the second word vector;
comparing the first similarity score to a threshold similarity score to determine whether the first text description matches the second text description; and
based on comparing the first similarity score to a threshold similarity score, generating for display, on a user interface, a recommendation.

2. The system of claim 1, wherein determining a first similarity score comprises:
comparing the first text description with each text description of the second plurality of text descriptions;
based on comparing the first text description with each text description of the second plurality of text descriptions, determining that the first text description does not match any of the text descriptions of the second plurality of text descriptions; and
in response to determining that the first text description does not match any of the text descriptions of the second plurality of text descriptions, determining the first similarity score.

3. The system of claim 1, wherein determining a first similarity score comprises:
generating, based on a comparison between the first word vector and each word vector of the second plurality of word vectors, a plurality of similarity scores;
determining that the first similarity score is higher than any other score of the plurality of similarity scores; and
in response to determining that the first similarity score is higher than any other score of the plurality of similarity scores, determining that the first similarity score should be compared with the threshold similarity score.

4. The system of claim 1, wherein a portion of the user interface indicates that the second text description is an adverse event of the vaccine and that the second text description does not appear on a label of the vaccine.

5. A method for using machine learning and natural language processing to determine which adverse events indicated in a database should be added to a vaccine label, comprising:
receiving, from a first database, adverse event data corresponding to a drug, wherein the adverse event data comprises a first plurality of text descriptions, wherein each text description of the first plurality of text descriptions indicates an adverse event associated with the drug;
generating, based on inputting the first plurality of text descriptions into a machine learning model, a first plurality of word vectors, wherein each word vector of the first plurality of word vectors corresponds to a text description of the first plurality of text descriptions;
receiving, from a second database, a second plurality of text descriptions, wherein each text description of the second plurality of text descriptions indicates a side effect;
generating a second plurality of word vectors, wherein each word vector of the second plurality of word vectors corresponds to a text description of the second plurality of text descriptions;
determining, based on a comparison of a first word vector of the first plurality of word vectors with a second word vector of the second plurality of word vectors, a first similarity score indicating a similarity between a first text description corresponding to the first word vector and a second text description corresponding to the second word vector;
comparing the first similarity score to a threshold similarity score to determine whether the first text description matches the second text description; and
based on comparing the first similarity score to the threshold similarity score, generating for display, on a user interface, a recommendation.

6. The method of claim 5, further comprising:
generating, based on an additional word vector of the first plurality of word vectors and the second word vector of the second plurality of word vectors, a second similarity score indicating a similarity level between an additional text description corresponding to the additional word vector and the second text description;
determining that the second similarity score fails to exceed the threshold similarity score;
based on determining that the second similarity score fails to exceed the threshold similarity score, generating a data structure comprising the additional text description and the second text description; and
storing the data structure in a queue for review by a medical professional.

7. The method of claim 6, wherein generating a data structure comprising the additional text description and the second text description comprises determining that the second similarity score exceeds a second threshold similarity score.

8. The method of claim 6, further comprising:
based on determining that the second similarity score fails to exceed the threshold similarity score, retrieving contextual information comprising an indication of symptoms experienced by a user associated with the additional text description, wherein the contextual information further comprises biographical information of the user; and
storing the contextual information in the data structure.

9. The method of claim 5, further comprising:
generating, based on a comparison of the second word vector with each vector of the first plurality of word vectors, a plurality of similarity scores;
determining that more than a threshold number of similarity scores of the plurality of similarity scores exceed the threshold similarity score; and
in response to determining that more than a threshold number of similarity scores of the plurality of similarity scores exceed the threshold similarity score, generating a recommendation indicating that the second text description should be added to a drug label associated with the drug.

10. The method of claim 5, further comprising:
comparing the first text description with each text description of the second plurality of text descriptions;
based on comparing the first text description with each text description of the second plurality of text descriptions, determining that the first text description does not match any of the text descriptions of the second plurality of text descriptions; and
in response to determining that the first text description does not match any of the text descriptions of the second plurality of text descriptions, determining the first similarity score.

11. The method of claim 5, determining a first similarity score comprises:
generating, based on a comparison between the first word vector and each word vector of the second plurality of word vectors, a plurality of similarity scores;
determining that the first similarity score is higher than any other score of the plurality of similarity scores; and
in response to determining that the first similarity score is higher than any other score of the plurality of similarity scores, determining that the first similarity score should be compared with the threshold similarity score.

12. The method of claim 5, wherein a portion of the user interface indicates that the second text description is an adverse event a patient experienced after taking the drug and that the second text description does not appear on a label of the drug.

13. A tangible, non-transitory, machine-readable medium for using machine learning and natural language processing to determine which adverse events indicated in a database should be added to a drug label, the medium storing instructions that when executed by one or more processors effectuate operations comprising:
receiving, from a first database, adverse event data corresponding to a drug, wherein the adverse event data comprises a first plurality of text descriptions, wherein each text description of the first plurality of text descriptions indicates an adverse event associated with the drug;
generating, based on inputting the first plurality of text descriptions into a machine learning model, a first plurality of word vectors, wherein each word vector of the first plurality of word vectors corresponds to a text description of the first plurality of text descriptions;
receiving, from a second database, a second plurality of text descriptions, wherein each text description of the second plurality of text descriptions indicates a side effect;
generating a second plurality of word vectors, wherein each word vector of the second plurality of word vectors corresponds to a text description of the second plurality of text descriptions;
determining, based on a comparison of a first word vector of the first plurality of word vectors with a second word vector of the second plurality of word vectors, a first similarity score indicating a similarity between a first text description corresponding to the first word vector and a second text description corresponding to the second word vector;
comparing the first similarity score to a threshold similarity score to determine whether the first text description matches the second text description; and
based on comparing the first similarity score to the threshold similarity score, generating for display, on a user interface, a recommendation.

14. The medium of claim 13, wherein the instructions, when executed by one or more processors, effectuate operations further comprising:
generating, based on an additional word vector of the first plurality of word vectors and the second word vector of the second plurality of word vectors, a second similarity score indicating a similarity level between an additional text description corresponding to the additional word vector and the second text description;
determining that the second similarity score fails to exceed the threshold similarity score;
based on determining that the second similarity score fails to exceed the threshold similarity score, generating a data structure comprising the additional text description and the second text description; and
storing the data structure in a queue for review by a medical professional.

15. The medium of claim 14, wherein generating a data structure comprising the additional text description and the second text description comprises determining that the second similarity score exceeds a second threshold similarity score.

16. The medium of claim 14, wherein the instructions, when executed by one or more processors, effectuate operations further comprising:
based on determining that the second similarity score fails to exceed the threshold similarity score, retrieving contextual information comprising an indication of symptoms experienced by a user associated with the additional text description, wherein the contextual information further comprises biographical information of the user; and
storing the contextual information in the data structure.

17. The medium of claim 13, wherein the instructions, when executed by one or more processors, effectuate operations further comprising:
generating, based on a comparison of the second word vector with each vector of the first plurality of word vectors, a plurality of similarity scores;
determining that more than a threshold number of similarity scores of the plurality of similarity scores exceed the threshold similarity score; and
in response to determining that more than a threshold number of similarity scores of the plurality of similarity scores exceed the threshold similarity score, generating a recommendation indicating that the second text description should be added to a drug label associated with the drug.

18. The medium of claim 13, wherein the instructions, when executed by one or more processors, effectuate operations further comprising:
comparing the first text description with each text description of the second plurality of text descriptions;
based on comparing the first text description with each text description of the second plurality of text descriptions, determining that the first text description does not match any of the text descriptions of the second plurality of text descriptions; and
in response to determining that the first text description does not match any of the text descriptions of the second plurality of text descriptions, determining the first similarity score.

19. The medium of claim 13, wherein the instructions for determining a first similarity score, when executed, effectuates operations further comprising:
generating, based on a comparison between the first word vector and each word vector of the second plurality of word vectors, a plurality of similarity scores;
determining that the first similarity score is higher than any other score of the plurality of similarity scores; and
in response to determining that the first similarity score is higher than any other score of the plurality of similarity scores, determining that the first similarity score should be compared with the threshold similarity score.

20. The medium of claim 13, wherein a portion of the user interface indicates that the second text description is an adverse event a patient experienced after taking the drug and that the second text description does not appear on a label of the drug.
